(19) United States
(12) Patent Application Publication
(10) Pub. No.: US 2016/0008529 A1
(43) Pub. Date: Jan. 14, 2016

(54) Peritoneal Dialysis System and Method

(71) Applicant: Josef C. A. Hoffman, (US)

(72) Inventor: Josef C. A. Hoffman, Irvine, CA (US)

(21) Appl. No.: 14/329,727

(22) Filed: Jul. 11, 2014

(51) Int. Cl. A61M 1/28; A61M 1/32

(52) U.S. Cl. CPC A61M 1/287; A61M 1/288; A61M 1/28; A61M 1/32

(57) ABSTRACT

A peritoneal dialysis machine that includes a base, a boiling vessel, a condenser, a fluid storage vessel, a sterilizing UV lamp, a fluid mixer, a dialysate cassette, a boiling vessel demineralizing system, and an optional fluid storage vessel rinsing system. The peritoneal dialysis machine automatically generates a predetermined volume of distilled water each day. Pre-weighed quantities of low-endotoxin dialysate chemicals are then mixed into the warm distilled water, and the resulting peritoneal dialysate is sterilized via exposure to ultraviolet radiation. These actions are complete by the patient's indicated bed time. The peritoneal dialysis machine then exchanges spent peritoneal dialysate in the user's peritoneal cavity with fresh peritoneal dialysate throughout the night, causing each infusion of fresh dialysate to dwell in the user's peritoneal cavity for a predetermined time. All spent fluids are routed from the peritoneal dialysis machine to a toilet or a sewer drain.

### FIELD OF THE INVENTION

The present invention relates to the generation of peritoneal dialysate, and to conducting peritoneal dialysis. The peritoneal dialysis machine of the present invention is particularly suitable for use in the patient's home, or in a nursing home.

### BACKGROUND OF THE INVENTION

The current world standard for peritoneal dialysis is Automated Peritoneal Dialysis (APD), wherein an automated cycler exchanges spent dialysate in the patient's peritoneal cavity with fresh dialysate, allowing the fresh dialysate to dwell in the peritoneal cavity for a predetermined period between each exchange. An end-stage renal patient will typically receive three to six dialysate exchanges per day, seven days a week. This method of APD has changed little in the past 30 years.

There are many U.S. patents associated with peritoneal dialysis, and the large majority of these patents are associated with a machine or a process that exchanges spent dialysate in the patient's peritoneal cavity, for fresh dialysate that was manufactured in a plant. Few U.S. patents are associated with a machine that generates fresh peritoneal dialysate, and then dialyzes the patient using the fresh dialysate. Some U.S. patents that are associated with such a machine include U.S. Pat. No. 7,892,423, U.S. Pat. No. 8,034,235, U.S. Pat. No. 8,206,578, U.S. Pat. No. 8,216,452, and U.S. Pat. No. 8,419,933 (all by Justin Rohde and William W. Han, for Baxter Healthcare and Baxter International). While these patents are associated with a machine that generates peritoneal dialysate and also dialyzes a patient, these patents focus on electrical power sources, power distribution, and power management for the peritoneal dialysis machine, more than on the machine itself.

The machine of the present invention was designed to meet all of the following requirements simultaneously: low operating cost, simple and easy to use in the home, reasonably low electrical current draw, maintain low bioburden and endotoxicity levels, and produce a dialysate that has a relatively neutral pH and almost no Glucose Degradation Products (GDPs). Several technologies exist that can purify water, such as carbon beds, deionization, distillation, water softening, and reverse osmosis. And several technologies exist that can sterilize fluids, such as heat, sterilizing ultraviolet radiation, Ozonation, Gamma radiation, and sterile filtration. Of these technologies, only distillation and sterilizing ultraviolet radiation can meet all of the requirements defined for the machine of the present invention. For this reason, these two technologies are specifically called out for the machine of the present invention.

There are five inherent disadvantages with the standard Automated Peritoneal Dialysis method. First, it is very costly for dialysate providers to manufacture up to 18 liters of dialysate per patient per day, and then ship it to each peritoneal dialysis patient's home. Second, existing dialysates have an acidic pH of 5.1 to 5.5, and they contain Glucose Degradation Products (GDPs). The acidic pH and the presence of GDPs make the dialysate non-biocompatible with the patient's peritoneum, and continuous contact with the non-biocompatible dialysate gradually degrades the peritoneum. Third, it is impractical for dialysate providers to offer a wide variety of dialysate formulae in order to accommodate each patient's individual blood chemistry. Fourth, patients dislike having to transport, handle and set up the floppy, heavy bags of dialysate every night. Finally, a great deal of storage space is required in the patient's home, for bags of fresh dialysate and for the large quantity of trash that is generated when using Automated Peritoneal Dialysis.

The present invention has none of the disadvantages described above. Because the machine of the present invention makes all of the required dialysate in the patient's home, the provider's cost is approximately one third that of the standard Automated Peritoneal Dialysis method. And the dialysate produced by the machine of the present invention is more biocompatible than plant-manufactured dialysate, because it has a pH of 6.2 to 7.0, and it contains virtually no GDPs.

When using the machine of the present invention, because each patient's dialysate is produced every evening, it is easy for a nephrologist to customize the calcium and/or sodium content for each patient's dialysate, to help treat hypo or hypercalcemia, or hypo or hypernatremia. If appropriate, it is also easy for a nephrologist to reduce the dialysate's glucose content to the point that the dialysate will hydrate the patient during dialysis, rather than cause the patient's body to shed water.

When using the machine of the present invention, because the water for each day's dialysate is supplied to the machine via a flexible tube, and because the spent fluids are routed from the machine to a toilet via a second flexible tube, bags of dialysate and bags for spent fluids are not needed or used. And because the daily consumables consist only of a compact cassette and small bottles of dialysate chemicals, only minimal storage space is required, and only a minimal amount of trash is generated.

### BRIEF SUMMARY OF THE INVENTION

The major assemblies of the machine of the present invention are a base, a still, a fluid storage tank, a cassette, a boiling vessel demineralization system, and a tank rinsing system. The still consists of a boiling vessel, a heating element, a temperature sensor, a demister, and a condenser. In the preferred embodiment, the still generates about 1.4 liters of distilled water per hour, and it draws about 8.7 Amps of current at 115 Volts. A large sterilizing low pressure mercury ultraviolet (UV) lamp is installed inside the fluid storage tank, and optional UV lamps and reflective covers can be mounted to the demister and the condenser. Tap water is supplied to the machine via a flexible tube, and a second flexible drain tube carries spent fluids from the machine to the nearest toilet or sewer drain.

In addition to a sterilizing UV lamp, the fluid storage tank includes a fluid mixer, a cooling water pad and a heating pad (to cool and then maintain the dialysate's temperature at 98.6° F.), ports for rinse water ingress and egress, ports for distilled water ingress and dialysate egress, an automatically locking/unlocking chemical addition port, a static rinse water distributor, a water electrical resistivity sensor, a fluid temperature probe, a narrow-band UV light sensor, and at least one fluid level sensor.

There are three sizes fluid storage tanks—10 liters, 14 liters and 18 liters. The 10 liter tank will accommodate volumes of 6, 7, 8, 9 and 10 liters of dialysate. The 14 liter tank will accommodate volumes of 11, 12, 13 and 14 liters of dialysate. The 18 liter tank will accommodate volumes of 15, 16, 17 and 18 liters of dialysate. Having more than one available tank size allows for a more compact tank if the patient's required daily volume of dialysate is less than 11 liters or less than 15 liters.

The dialysis chemicals consist of low-endotoxin powdered dextrose monohydrate (also known as glucose), powdered low-endotoxin salts, and low-endotoxin sodium lactate (solution or powder). The salts consist of sodium chloride, magnesium chloride hexahydrate, and calcium chloride dihydrate. The quantities of these chemicals are specific to each patient's prescription, and they come in bottles that are designed to mate with the tank's chemical fill port. A boiling vessel demineralization solution is also required. This solution is a weak acid, such as distilled white vinegar.

Each day, the machine of the present invention generates a prescribed volume of warm, sterile peritoneal dialysate, which is timed to be ready for use at the patient's indicated bed time. The machine tracks the volumes of dialysate that are pumped into and out of the patient during each exchange cycle. Because dialysis intentionally causes the patient to shed excess water, the volume of spent dialysate removed from the patient will almost always be greater than the volume of fresh dialysate that is pumped in. After each exchange cycle, the machine calculates the progressive cumulative difference between the two volumes. The total difference for the entire evening is the amount of excess water that was withdrawn from the patient by that dialysis session. This information can be displayed on the display screen. These calculations would be reversed in the rare cases of patients who need the dialysis procedure to hydrate them.

The machine of the present invention automatically demineralizes the boiling vessel each evening, and it automatically rinses the fluid storage tank each morning.

The machine of the present invention continuously tracks the number of days remaining until the next required periodic maintenance activity. The maintenance activities involve replacing a few components. No tools or special skills are required to complete the activities. When a maintenance activity due date is a few days away, the machine begins informing the user every day, verbally and on the display screen. On the maintenance activity's due date, the machine verbally (and on the display screen) prompts the patient and instructs him how to perform the activity. The tap water supply tube and the drain tube should be replaced once per quarter (or possibly a different elapsed time), and the sterilizing UV lamp(s) and the boiling vessel heating element should be replaced once a year (or possibly a different elapsed time).

### BRIEF DESCRIPTION OF THE DRAWINGS

FIG. 1 depicts one embodiment of the dialysis machine, as seen from the front

FIG. 2 depicts one embodiment of the fluid storage tank as seen from above, not including the condenser and the fluid mixer motor

FIG. 3 depicts one embodiment of the fluid storage tank, as seen from below

FIG. 4 depicts one embodiment of a cutaway view of the cassette, as seen from above

FIG. 5 depicts one embodiment of the base as seen from above, not including the fluid storage tank, the boiling vessel, the cassette, and the rinse solution containers

FIG. 6 depicts one embodiment of the components housed in the base

FIG. 7 depicts one embodiment of an electrical diagram of the dialysis machine

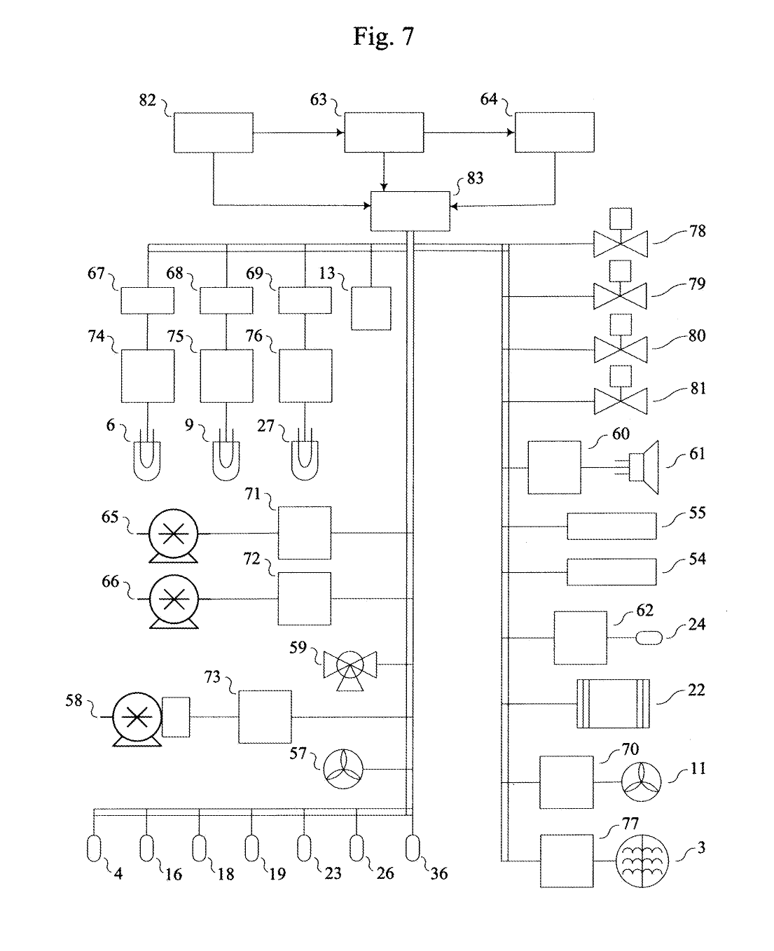

FIG. 8 depicts one embodiment of a fluid flow diagram of the dialysis machine

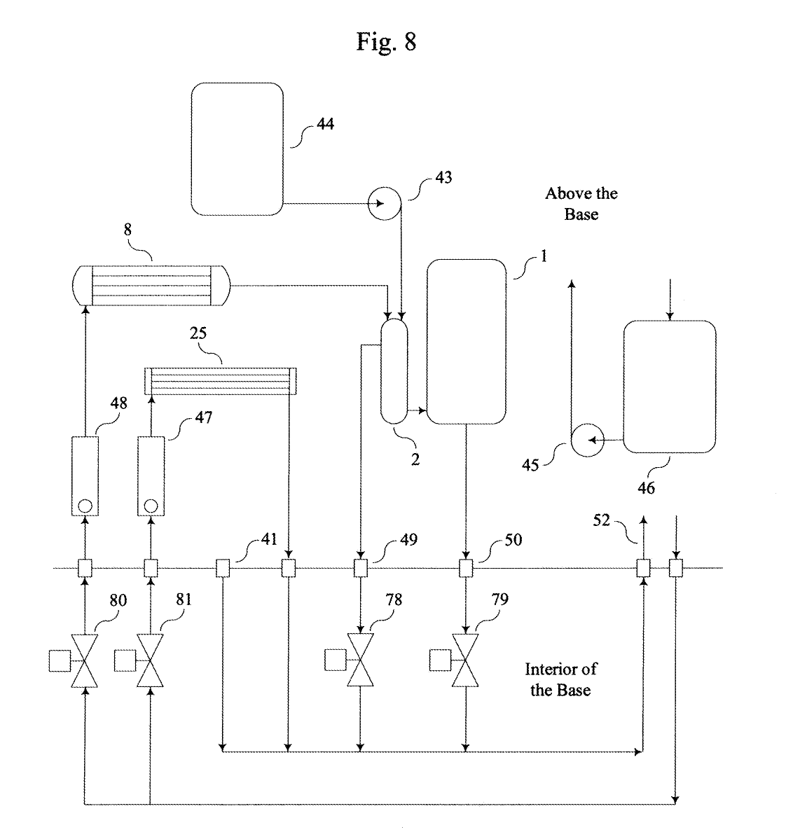

### DETAILED DESCRIPTION OF THE INVENTION

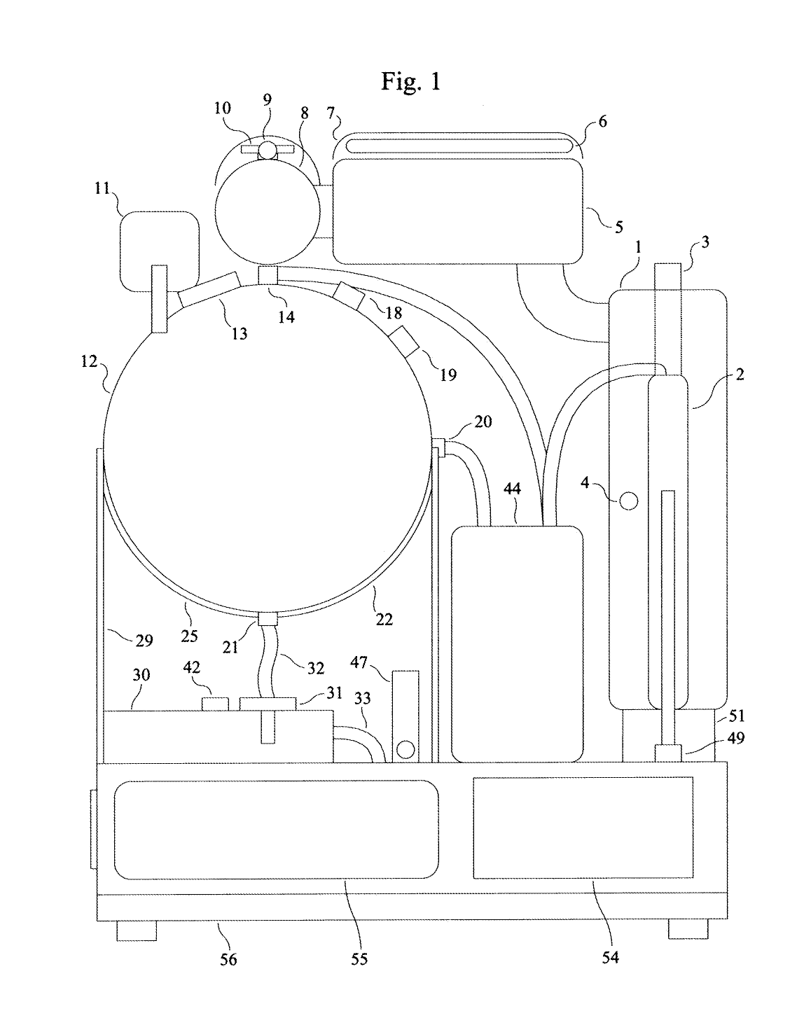

As seen in FIG. 1, boiling vessel 1 is located at the right rear base 56. It is composed of Pyrex glass, and tap water enters it via constant level tube 2. The boiling vessel might include a temperature insulation jacket. In one embodiment, boiling vessel heating element 3 inserts into the top of the boiling vessel, creating an air tight seal when installed. In an alternate embodiment, the boiling vessel is enclosed in a compact microwave heater. The boiling vessel generates steam during operation, which travels into demister 5. Boiling vessel drain port 50 is located directly below the boiling vessel.

As seen in FIG. 1, constant level tube 2 is attached to boiling vessel 1. It is composed of Pyrex glass. During operation, tap water flows from condenser 8 to the constant level tube, which maintains a constant water level in boiling vessel 1. Water that does not flow into the boiling vessel, continuously overflows into the constant level tube's spill port. Constant level tube drain port 49 is located below the constant level tube.

As seen in FIG. 1, in one embodiment, boiling vessel heating element 3 inserts into the top of the boiling vessel. This assembly includes a quartz tube that seals air tight with the boiling vessel, and yet is easily accessible and removable. In an alternate embodiment, the boiling vessel is enclosed in a compact microwave heater. The heating element uses 115 Volts AC and draws about 8.7 Amps, thereby using 1000 Watts (or possibly a different current and power), allowing the boiling vessel to distill 1.4 liters of tap water per hour (or possibly a different rate). Optional current sensor 77 continuously measures the current drawn by the boiling vessel heating element when it is energized, and an alarm can be generated if the current is outside its alarm limits.

As seen in FIG. 1, boiling vessel temperature sensor 4 is mounted close to the wall of boiling vessel 1, at or near the water level during operation. The temperature sensor might be a thermostat that breaks a circuit when a high temperature is sensed, or it might be an NTC type thermistor. In either case, an alarm is generated if the boiling vessel's temperature exceeds 220° F. (or possibly a different alarm temperature). The most likely cause of a high temperature is the boiling vessel heating element being mistakenly energized when little or no water is in the boiling vessel.

As seen in FIG. 1, demister 5 is located between boiling vessel 1 and condenser 8. If a UV light is mounted on the demister, then the demister is composed of quartz glass. If a UV light is not mounted on the demister, then the demister is composed of Pyrex glass (or possibly another type of glass). The demister removes tiny floating water droplets from the steam that comes from the boiling vessel. The demisted steam then passes through to the condenser.

As seen in FIG. 1, demister UV lamp 6 is mounted on the demister. The lamp is a low pressure mercury vapor type, and it is about the same length as the demister. The lamp is powered by ballast 67, and it uses about 9 Watts of power (or possibly a different amount). Optional current sensor 74 continuously measures the current drawn by the demister UV lamp when it is energized, and an alarm is generated if the current is outside its alarm limits. The demister UV lamp's bulb is doped with heavy metals (or other agents) that absorb UV light having wavelengths shorter than 240 nm, to prevent the lamp from generating ozone from the oxygen in the surrounding air. The lamp is easily removable for replacement. The demister UV lamp is an optional component. In an alternate embodiment, the demister UV lamp does not exist.

As seen in FIG. 1, reflective UV lamp covers 7 surround demister 5 and demister UV lamp 6, and condenser 8 and condenser UV lamp 9. The interior surfaces of the covers are highly polished, and they reflect UV light onto all surfaces of the demister and condenser. The covers are opaque, in order to protect the surrounding environment from exposure to UV light. The covers are installed only if the optional UV lamps are installed on the demister and the condenser. In an alternate embodiment, the reflective UV lamp covers do not exist.

As seen in FIG. 1, condenser 8 is mounted above fluid storage tank 12. The condenser condenses the steam coming from demister 5, and delivers the condensate to the fluid storage tank via tank distilled water fill port 17. Tap water enters the condenser from tank cooling water flow controller 47, and water flows from the condenser to constant level tube 2. If a UV light is mounted on the condenser, then the condenser is composed of quartz glass. If a UV light is not mounted on the condenser, then the condenser is composed of Pyrex glass (or possibly another type of glass). The condenser is oriented to be about 18 degrees from horizontal (or possibly a different angle), such that condensed water flows towards the tank distilled water fill port. Condenser vent filter 10 is connected to the condenser vent, which is located at the highest point on the condenser. The vent filter filters gases that are flowing into and out of the condenser.

As seen in FIG. 1, condenser UV lamp 9 is mounted on the condenser. The lamp is a low pressure mercury vapor type, and it is about the same length as the condenser. The lamp is powered by condenser UV lamp ballast 68, and it uses about 24 Watts of power (or possibly a different amount). Optional current sensor 75 continuously measures the current drawn by the UV lamp when it is energized, and an alarm is generated if the current is outside its alarm limits. The UV lamp's bulb is doped with heavy metals (or other agents) that absorb UV light having wavelengths shorter than 240 nm, to prevent the lamp from generating ozone from the oxygen in the surrounding air. The lamp is easily removable for replacement. The condenser UV lamp is an optional component. In an alternate embodiment, the condenser UV lamp does not exist.

As seen in FIG. 1, condenser vent filter 10 is connected to the condenser at its highest point. The pore diameter in the filter membrane is 25 μm (or possibly a different pore diameter). The filter allows egress of volatile gases released during distillation of tap water, and also allows ambient air to enter the condenser as the fluid storage tank gradually drains throughout the night. The condenser vent filter is an optional component. In an alternate embodiment, the condenser vent filter does not exist

As seen in FIG. 1, fluid mixer and motor 11 is mounted on fluid storage tank 12, at the top left rear. The propeller shaft and blades are made of 316 stainless steel, and they are polished, electropolished, and passivated. The shaft penetrates the tank wall 45° from vertical (or possibly a different angle), such that the propeller propels fluid in the tank downward and toward the front of the tank, when rotating. The motor uses 115 Volts and it draws 50 Watts of power (or possibly a different power). Optional current sensor 70 continuously measures the current drawn by the mixer motor when it is energized, and an alarm is generated if the current is outside its alarm limits. The junction of the shaft and the propeller blades is smooth, without any gaps, cracks, or crevasses. The motor is very quiet during operation.

As seen in FIGS. 1, 2 and 3, fluid storage tank 12 is mounted above the left half of base 56. It is made of 316 stainless steel, and it is polished, electropolished and passivated on its interior and exterior. The tank is supported by tank support brackets 29. The tank is available in three sizes: 10 liters, 14 liters, and 18 liters (or possibly different volumes). The 10 liter tank will accommodate volumes of 6, 7, 8, 9 and 10 liters of dialysate. The 14 liter tank will accommodate volumes of 11, 12, 13 and 14 liters of dialysate. The 18 liter tank will accommodate volumes of 15, 16, 17 and 18 liters of dialysate. The different tank sizes permit optimal matching of tank size with the daily volume of dialysate required by each patient. The tank and its fittings are designed such that almost no shadows are cast from the light coming from the tank UV lamp 27, which is located in the tank. The welds of the fittings in the tank walls are radiused, polished smooth, and free of tiny cracks or crevices. The tank body and the fittings in/on the tank are electropolished and passivated after being welded and polished. The fluid storage tank is mounted at a slight slope, such that fluid naturally drains towards tank dialysate exit port 21.

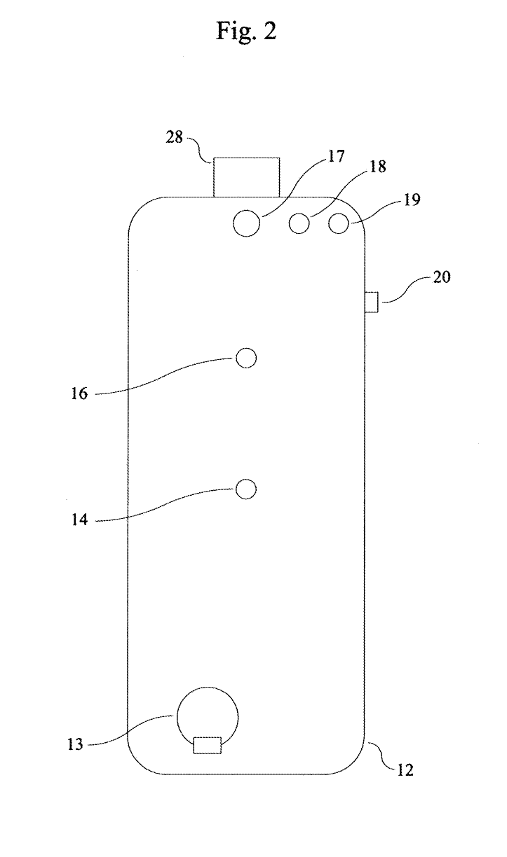

As seen in FIGS. 1 and 2, tank chemical fill port and locking mechanism 13 is located at the front end of the tank, near the top. The port is designed to have the minimum possible surface area, and it is designed such that all interior surfaces are bathed in UV light from tank UV lamp 27. Each evening, when the required quantity of distilled water has been generated and then cooled to 98.6° F., the port's stainless steel outer cap is automatically unlocked and the patient is alerted to add chemicals to the distilled water. The patient removes and discards the old plastic port cap, pours powdered dextrose, powdered salts and sodium lactate (solution or powder) into the tank, snaps a fresh sterilized plastic cap onto the port, and then pivots the stainless steel outer cap closed, onto the plastic cap. The patient then presses the “Confirm Addition of Chemicals” button. This causes the port cap's locking mechanism to automatically lock the outer cap closed until another batch of distilled water is ready for chemicals to be added to it, approximately 24 hours later.

As seen in FIGS. 1 and 2, tank rinse water entry port 14 is located in the top center of wall of fluid storage tank 12. The port is made of 316 stainless steel, and it is configured as a female locking luer (or possibly a different configuration). The tube from tank rinse water container assembly 46 connects to this port. In an alternate embodiment, the tank is never rinsed, so the tank rinse water entry port does not exist.

Tank rinse water distributor 15 is welded to the top interior of fluid storage tank 12, approximately one inch directly below rinse water entry port 14. The distributor has the shape of a “Hershey's Kiss”, it has no moving parts, and it is made of 316 stainless steel. When the tank is being rinsed, rinse water enters the port as a fast-moving stream that flows vertically downward onto the distributor. The distributor diverts the rinse water onto the upper interior surfaces of the tank, as well as the quartz tube and the mixer propeller shaft. The rinse water then flows down the walls of the tank, and is pumped out of the tank via tank dialysate exit port 21. The distributor is designed so that reflected UV light can reach its upper face, as well as the interior of the tank wall that is above the distributor. In an alternate embodiment, the tank is never rinsed, so the tank rinse water distributor does not exist.

As seen in FIG. 2, tank UV light sensor 16 is located in the top rear wall of fluid storage tank 12. The sensor is a Gallium Nitride photodiode (or possibly another type) that faces tank UV lamp 27. The sensor is sensitive to light having wavelengths of 220 nm to 280 nm. The sensor continuously monitors the UV light intensity in the tank while the tank UV lamp is energized. An alarm is generated if the UV light intensity goes outside alarm limits. The sensor is an optional component. In an alternate embodiment, the tank UV light sensor does not exist.

As seen in FIG. 2, tank distilled water fill port 17 is located in the top rear wall of fluid storage tank 12. It is made of 316 stainless steel. The condenser's drain port is connected to the tank distilled water fill port.

As seen in FIGS. 1 and 2, tank point level sensors 18 and 19 are located in the rear wall of fluid storage tank 12. These sensors sense when distilled water in the tank has risen to the level of the sensor. During operation, distilled tap water gradually fills the tank until the water level reaches one of these sensors, at which point the distilling process automatically stops. The vertical locations of the sensors correspond to specific fluid volumes in the tank. Four or five point level sensors are installed in each fluid storage tank, corresponding to four of five specific fill volumes. This configuration allows the machine to generate the exact daily quantity of dialysate that has been prescribed for the patient.

As seen in FIGS. 1 and 2, tank rinse water exit port 20 is located in the wall of fluid storage tank 12, at the vertical midpoint near the rear of the tank. The port is made of 316 stainless steel, and it is configured as a female locking luer (or possibly a different configuration). A tube from tank rinse water container assembly 46 connects to this port. During distillation, when the distilled water level reaches the level of this port, distilled water flows through the port into the rinse water container until the container is full. In an alternate embodiment, the tank is never rinsed, so the tank rinse water exit port does not exist.

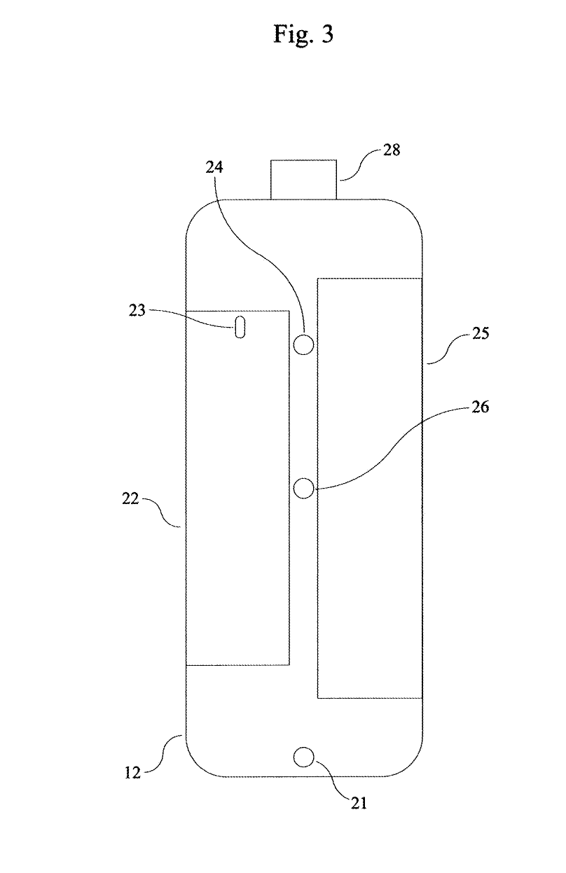

As seen in FIGS. 1 and 3, tank dialysate exit port 21 is located in the bottom front wall of fluid storage tank 12. The port is made of 316 stainless steel, and it is configured as a female locking luer (or possibly a different configuration). Cassette dialysate supply tube 32 connects to this port.

As seen in FIGS. 1 and 3, tank heating pad 22 is attached to the exterior wall of fluid storage tank 12, on a lower quadrant. During dialysis, this electrical-resistance-type heating pad is energized as needed to maintain the dialysate temperature at 98.6° F. The dialysate temperature is measured by tank fluid temperature probe 26. The heating pad is made out of silicone (or possibly a different material). Optional heating pad temperature sensor 23 is attached to the heating pad.

As seen in FIG. 3, heating pad temperature sensor 23 is attached to tank heating pad 22. The temperature sensor is an NTC type thermistor (or possibly a different sensor). The temperature of the heating pad is continuously monitored while the pad is energized, to prevent the temperature of the heating pad from exceeding approximately 120° F. (or possibly a different temperature). The heating pad temperature sensor is an optional component. In an alternate embodiment, the heating pad temperature sensor does not exist.

As seen in FIG. 3, water electrical resistivity sensor 24 is located in the bottom rear wall of fluid storage tank 12. The sensor is wired to water electrical resistivity circuit board 62. During tap water distillation, the sensor continuously monitors the electrical resistivity of the distilled water in fluid storage tank 12. Distilled water of high purity has a high electrical resistivity, and vice versa. In addition, the electrical resistivity of a given sample of purified water is a function of its temperature. The electrical resistivity of the distilled water generated by the present invention is at least 1.0 Mohm-cm at 175° F., 3.0 Mohm-cm at 120° F., and 5.0 Mohm-cm at 98.6° F. In the present invention, the water resistivity alarm logic simultaneously considers both the measured resistivity and the temperature of the distilled water, on a continuous basis. An alarm signal is generated if the resistivity of the distilled water falls below the alarm resistivity limit that is appropriate for the temperature. During operation, this sensor is energized only after the distilled water level is high enough to cover the probe, and it is de-energized just before the dialysate chemicals are added to the distilled water. The water electrical resistivity sensor is an optional component. In an alternate embodiment, the water electrical resistivity sensor does not exist.

As seen in FIGS. 1 and 3, tank cooling water pad 25 is attached to the exterior wall of fluid storage tank 12, on a lower quadrant. During the distillation and cool down phases, tap water flows through the cooling water pad until tank fluid temperature probe 26 indicates that the distilled water has been cooled to 98.6° F. Tap water is fed to the cooling water pad from tank cooling water flow controller 47, and water flows from the cooling water pad, to drain tube 52.

As seen in FIG. 3, tank fluid temperature probe 26 is located in the bottom center wall of fluid storage tank 12. It is made of polished and passivated 316 stainless steel, and it extends ½ inch into the fluid storage tank (or possibly a different length). The tank fluid temperature probe contains a NTC type thermistor (or possibly a different sensor). During operation, the fluid temperature probe continuously monitors the temperature of the fluid in the tank.

Tank UV lamp 27 is located in fluid storage tank 12, inside tank UV lamp quartz tube 28. It is mounted in the tank's rear wall and it runs horizontally down the entire length of the tank. The UV lamp can be located half way up the tank, or it can be located near the ceiling of the tank, above the fluid level. The lamp is a low pressure mercury vapor type. The lamp is powered by tank UV lamp ballast 69, and it uses about 35 Watts of power (or possibly a different amount). Current sensor 76 continuously measures the current drawn by the UV lamp when the lamp is energized, and an alarm is generated if the current is outside its alarm limits. The UV lamp's bulb is doped with heavy metals (or other agents) that absorb UV light having wavelengths shorter than 240 nm, to prevent the lamp from generating ozone from the oxygen in the surrounding air and water. The lamp is easily removable for replacement.

As seen in FIGS. 2 and 3, tank UV lamp quartz tube 28 is located inside fluid storage tank 12. It is mounted in the tank's rear wall and it runs horizontally down the entire length of the tank. The UV lamp quartz tube can be located half way up the tank, or it can be located near the ceiling of the tank, above the fluid level. The tube keeps fluids from contacting tank UV lamp 27, which is mounted inside the tube. It also allows the tank to remain water tight while the UV lamp is removed. The end of the tube near the front of the tank, is closed off. The tube is made of quartz because other types of glass absorb excessive amounts of UV light.

As seen in FIG. 1, tank support brackets 29 are attached to the base, and they hold fluid storage tank 12 in position. They are made of passivated 316 stainless steel or anodized aluminum, and strips of felt (or other similar protective material) are attached to the upper faces of the two semi-circular sections. The rear bracket is slightly taller than the front bracket, in order to tilt the tank slightly towards tank dialysate exit port 21. There are three sizes of tank support brackets, to accommodate the three sizes of fluid storage tank.

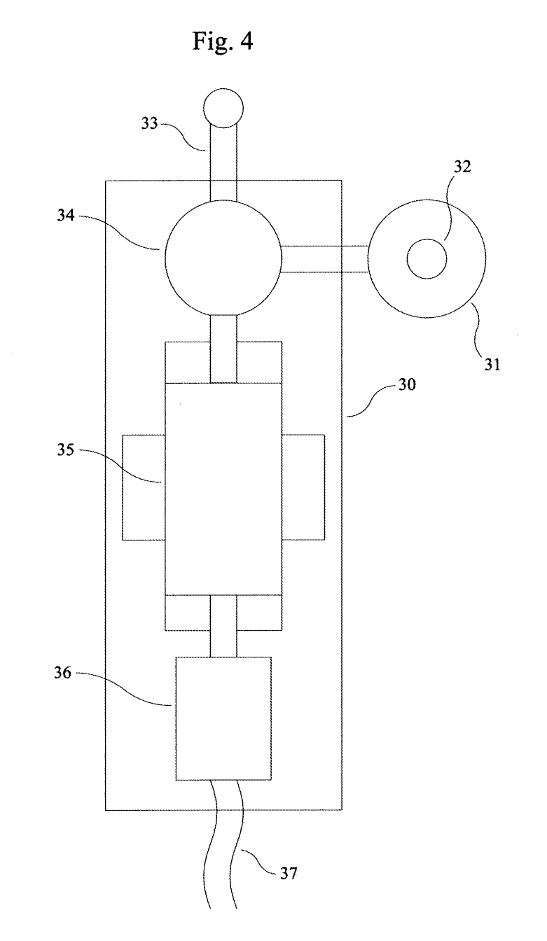

As seen in FIGS. 1 and 4, cassette 30 pumps dialysate from fluid storage tank 12 to the patient, and from the patient to dialysate drain port 41. In one embodiment, the cassette includes dialysate filter 31, dialysate supply tube 32, dialysate drain tube 33, cassette valve 34, dialysate pump occlusion bed 35, dialysate pressure sensor 36, and dialysate tube 37. In an alternate embodiment, the cassette contains a different pumping mechanism other than the dialysate pump occlusion bed. In yet another embodiment, the cassette contains a different mechanism for detecting the stoppage of dialysate flow, other than the dialysate pressure sensor. In yet another embodiment, the cassette contains two in-line check valves, which take the place of the cassette valve. The cassette is initially sterile, and kept in a sterile pouch until use. A fresh cassette is installed each day. Installing a cassette consists of clamping it into position on base 56 using cassette hold down fixture 42. The pump rotor mounting blocks on the base serve as alignment guides for the cassette. All of the wetted components in the cassette are biocompatible. The controller detects the presence of a cassette by electronically sensing the presence of the cassette dialysate pressure sensor (or possibly a different method). An alarm will be generated if a used cassette is not promptly replaced by a fresh cassette each morning, and an alarm will be generated if no cassette is installed for longer than 20 seconds (or possibly a different elapsed time).

As seen in FIGS. 1 and 4, cassette dialysate filter 31 is in fluid communication with cassette valve 34. It has a hydrophilic filter membrane, 25 μm pores (or possibly a different pore size), and a surface area of 2 inch² (or possibly a different surface area). The filter membrane is biocompatible and non-shedding, and it does not weaken or change its pore size after being continuously wetted for 9 hours. The pressure drop across the filter membrane is less than 0.25 psig (or possibly a different pressure drop) at 100 ml/minute dialysate flow rate. The filter housing has minimal internal “dead” volume.

As seen in FIGS. 1 and 4, cassette dialysate supply tube 32 is connected to cassette dialysate filter 31. When a fresh cassette is installed, the user will connect this tube to tank dialysate exit port 21. The tube terminates in a male luer lock connector (or possibly a different connector).

As seen in FIGS. 1 and 4, cassette dialysate drain tube 33 is connected to cassette valve 34. The dialysate drain tube terminates in a male self-sealing quick disconnect type connector (or possibly a different type). When a cassette is installed, the user connects this tube to dialysate drain port 41.

As seen in FIG. 4, cassette valve 34 is a three-way valve. It can be rotated 90° clockwise or counterclockwise. One position allows fluid communication between the patient and the fluid storage tank, and the other position allows fluid communication between the patient and dialysate drain port 41. When a cassette is installed, this valve aligns with cassette valve actuator 40, which is located directly under the cassette valve, in the top face of base 56. In another embodiment, the cassette valve is replaced by two in-line check valves.

As seen in FIG. 4, in one embodiment, dialysate pump occlusion bed 35 consists of a length of rubber tubing inside a semi-circular plastic occlusion bed. The occlusion bed holds the tube in a semi-circular curve, covering approximately 135° of arc (or possibly a different arc). When a cassette is installed, the rubber tube is squeezed between the occlusion bed's lower surface and the rollers in dialysate pump rotor 39, which is located directly under the pump occlusion bed, in the top face of base 56. The dialysate pump rotor pushes dialysate through the rubber tube in either direction, depending on the rotor's direction of rotation. In order to function correctly, the distance between the outermost surface of the pump rollers and the lower surface of the pump occlusion bed needs to be 1.6 times the wall thickness of the rubber tube. In an alternate embodiment, the dialysate pump occlusion bed is replaced by a different pumping mechanism.

As seen in FIG. 4, in one embodiment, cassette dialysate pressure sensor 36 is located immediately next to dialysate pump occlusion bed 35. This sensor monitors the pressure of the fresh or spent dialysate that is being pumped to or from the patient. The pressure sensor is primarily used to detect a vacuum step change as spent dialysate is being pumped out of the patient's peritoneal cavity. A vacuum step change during dialysate evacuation indicates that all of the spent dialysate has been removed from the patient's peritoneal cavity. When a cassette is installed, the cassette dialysate pressure sensor presses against electrical contacts 38 that are located directly under the pressure sensor, in the top face of base 56. This allows the pressure sensor to electronically communicate with control and input/output circuit boards 83. In an alternate embodiment, the cassette dialysate pressure sensor is replaced by a different mechanism for detecting dialysate flow stoppage.

As seen in FIG. 4, dialysate tube 37 is connected to cassette dialysate pressure sensor 36. This flexible one-lumen tube is ten feet long (or possibly a different length), and it allows fresh dialysate to be pumped to the patient and spent dialysate to be pumped from the patient. The connector at the free end of the dialysate tube is a standard PD connector that has a removable air-tight protective cap. In order to maintain its sterility, the patient removes the cap and attaches the connector to his Tenckhoff catheter only immediately before dialysis begins.

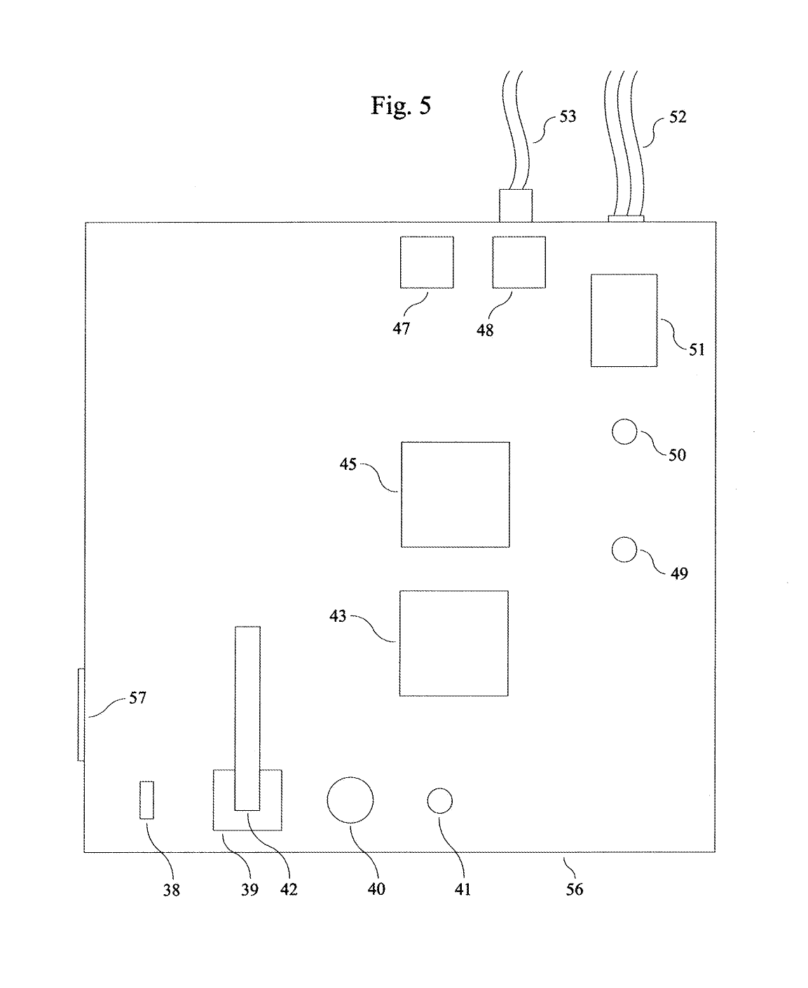

As seen in FIG. 5, in one embodiment, electrical contacts for the cassette dialysate pressure sensor 38 are located on the top face of base 56, near the left front. These spring-loaded contacts touch contacts in cassette dialysate pressure sensor 36 when cassette 30 is clamped into position. This allows electronic communication between the cassette dialysate pressure sensor and control and input/output circuit boards 83. In an alternate embodiment, the electrical contacts are for a mechanism that detects dialysate flow stoppage, other than a pressure sensor.

As seen in FIG. 5, in one embodiment, dialysate pump rotor 39 is located in the top face of base 56, near the left front. The rotor includes four rollers and it has a 1.25″ diameter (or possibly another diameter). During operation, the rotor rotates at 215 RPM (or possibly a different RPM) to pump dialysate at 100 ml/minute (or possibly a different flow rate). When the rotor rotates in one direction, spent dialysate is pumped from the patient, and when the rotor rotates in the opposite direction, fresh dialysate is pumped to the patient. The rotor and its shaft are made of aluminum or 316 stainless steel, and its rollers are made of plastic. An attached beveled gear meshes at a right angle with an identical gear that is mounted on the drive shaft of dialysate pump motor 58. The motor is mounted to the underside of the top face of base 56, under the dialysate pump rotor. An elastic o-ring can be stretched around the rollers to reduce roller noise during operation. Needle bearings or ball bearings support the rotor shaft at both ends. In an alternate embodiment, the dialysate pump rotor is replaced by a different pumping mechanism.

As seen in FIG. 5, cassette valve actuator 40 is located in the top face of base 56, next to dialysate pump rotor 39. The actuator is mounted on the gearbox output shaft of cassette valve motor 59, which is mounted to the underside of the top face of base 56, directly under the cassette valve actuator. When cassette 30 is clamped in place, the cassette valve actuator meshes with cassette valve 34. The cassette valve motor can then rotate the cassette valve 90 degrees clockwise or counterclockwise, as needed. A cassette valve rotation takes about two seconds (or possibly another period). In an alternate embodiment, the cassette valve actuator is not required, and therefore does not exist.

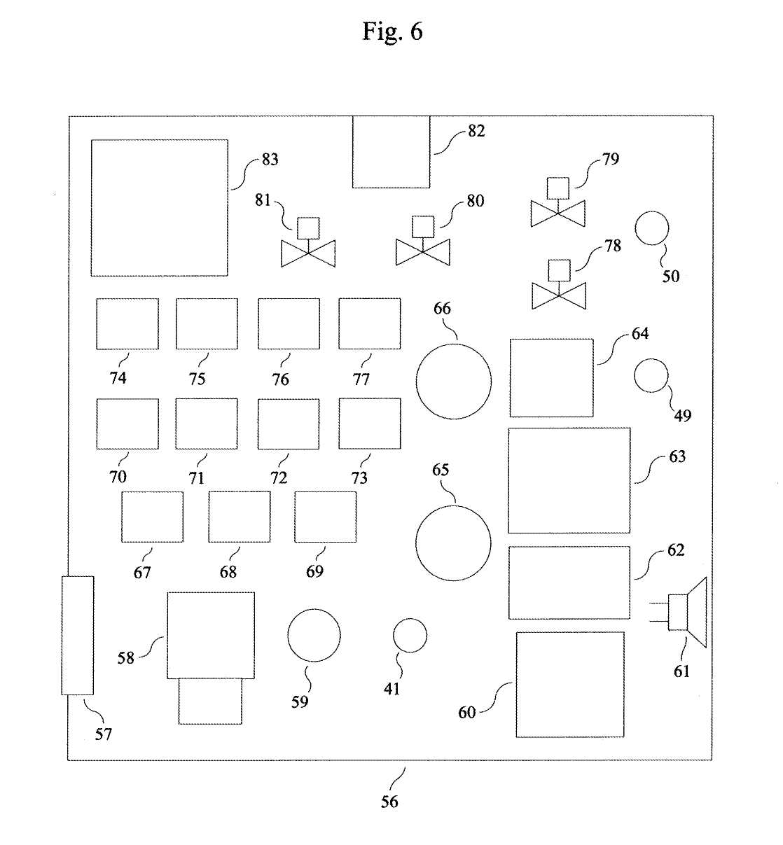

As seen in FIGS. 5 and 6, dialysate drain port 41 is located in the top face of base 56, next to cassette valve actuator 40. The port is in fluid communication with drain tube 52. When spent dialysate is being pumped from the patient, the spent dialysate is routed to this port. And when fluid storage tank 12 is being rinsed, the spent rinse water is routed to this port. The dialysate drain port is a female self-sealing quick disconnect type connector (or possibly a different type), and it is mounted facing vertically upward.

As seen in FIGS. 1 and 5, cassette hold down fixture 42 is located on the top face of base 56, near the left front. The fixture holds the cassette firmly in place during operation. The hold down fixture can be any type of clamping mechanism.

As seen in FIG. 5, boiling vessel demineralization solution pump 43 is mounted on the top surface of base 56, near the center. It can be a peristaltic pump with a pivoting “easy loading” pump head, or possibly a different type of pump. Boiling vessel demineralization solution container assembly 44 sits on the base near the pump, with its tube running to the pump. The boiling vessel is automatically demineralized each night, immediately after the distillation phase has been completed. During demineralization of boiling vessel 1, the pump pumps 545 ml (or possibly a different quantity) of demineralization solution at about one liter per minute (or possibly a different rate), from the demineralization solution container assembly, to constant level tube 2. This can be repeated, if necessary.

As seen in FIG. 1, boiling vessel demineralization solution container assembly 44 is a four liter (or possibly a different volume) plastic vessel that includes a flexible tube attached near its bottom. The tube goes to boiling vessel demineralization solution pump 43, and it continues to the top of constant level tube 2. The boiling vessel demineralization solution container assembly sits on the base, next to the boiling vessel demineralization solution pump. It is refilled with demineralization solution once per week.

As seen in FIG. 5, tank rinse water pump 45 is mounted on the top surface of base 56, near the center. It can be a peristaltic pump with a pivoting “easy loading” pump head, or possibly a different type of pump. Tank rinse water container assembly 46 sits on the base near the pump, with its tube running to the pump. Fluid storage tank 12 is semi-automatically rinsed each morning, after the evening's dialysis has been completed. During tank rinsing, the rinse water pump pumps one liter of distilled water (or possibly a different quantity) at one about liter per minute (or possibly a different rate), from the rinse water container to tank rinse water entry port 14. This is an optional component. In an alternate embodiment, the tank is never rinsed, so the tank rinse water pump does not exist.

Tank rinse water container assembly 46 is a one liter (or possibly a different volume) plastic vessel that has a short flexible tube attached at its top, and a longer flexible tube attached near its bottom. The short flexible tube connects to tank rinse water exit port 20. The longer tube goes to tank rinse water pump 45, and it continues to tank rinse water entry port 14. Both tubes terminate in a male luer lock connector (or possibly a different connector). The container is automatically filled with distilled water during the distillation phase. The tank rinse water container sits on the base, next to the tank rinse water pump. It is replaced daily with a sterilized assembly, and it is packaged in a sterile pouch. This is an optional assembly. In an alternate embodiment, the tank is never rinsed, so the tank rinse water container assembly does not exist.

As seen in FIG. 1, tank cooling water flow controller 47 is mounted on the top surface of base 56, at the rear. It is a manually adjustable metering valve and flow rate indicator that controls the flow rate of tap water through tank cooling water pad 25. The cooling water flow rate is maintained at about 15 liters per hour (250 ml/minute, or possibly a different flow rate). The exact adjustment will depend on the pressure of the tap water in the patient's home.

Condenser water flow controller 48 is mounted on the top surface of base 56, at the rear. It is a manually adjustable metering valve and flow rate indicator that controls the flow rate of tap water through condenser 8. The cooling water flow rate is maintained at about 15 liters per hour (250 ml/minute, or possibly a different flow rate). The exact adjustment will depend on the pressure of the tap water in the patient's home.

As seen in FIGS. 1 and 5, constant level tube drain port 49 is located in the top of the base, below constant level tube 2. It is a female self-sealing quick disconnect type connector (or possibly a different type), that it mounted to face vertically upward. The port allows the boiling vessel to be easily removed for replacement. It also allows the top panel of the base to be removed from the rest of the base, for conducting maintenance, repairs, or upgrades.

As seen in FIG. 5, boiling vessel drain port 50 is located in the top of the base, below boiling vessel 1. It is a female self-sealing quick disconnect type connector (or possibly a different type), that it mounted to face vertically upward. The port allows the boiling vessel to be easily removed for replacement. It also allows the top panel of the base to be removed from the rest of the base, for conducting maintenance, repairs, or upgrades.

As seen in FIGS. 1 and 5, boiling vessel support stand 51 is mounted to base 56, at the right rear. The stand holds boiling vessel 1, and it holds boiling vessel temperature sensor 4 such that it is almost in contact with the boiling vessel.

As seen in FIG. 5, tap water supply tube and drain tube 52 plug into two ports located in the back panel of base 56. They are flexible tubes that can be at least 50 feet long, with inner diameters of ⅛″ and ¼″, respectively (or possibly different inner diameters). The two tubes can be attached to each other along their lengths, or not. The tap water supply tube has a male self-sealing quick disconnect connector (or possibly a different type) at both ends. The drain tube has a male self-sealing quick disconnect connector (or possibly a different type) at one end, and a curved rigid plastic tube at other end, that hooks onto the lip of a toilet bowl. The connectors on the two tubes' end are colored, shaped, and/or labeled such that they can be easily distinguished from one another.

As seen in FIG. 5, power cord 53 is a 15 Amp grounded power cord that has a NEMA 5-15P plug at one end and an IEC 60320-1-C13 plug at the other end. An alternate style of plugs can be used. The power cord is ten feet long (or possibly a different length).

As seen in FIG. 1, LCD display screen 54 is located on the right half of the front control panel of base 56. The LCD display screen is back lit, which energizes for 60 seconds if any control pad button is pressed. The screen can be black and white, or color. It can be alpha-numeric or graphics type. Its electronics can be sealed against fluids, or not. Instructional and alarm messages are displayed on this screen as needed. Operational information and instructional and alarm messages that can be displayed, are given in paragraphs [0085], [0086], and [0087] below.

As seen in FIG. 1, control pad 55 is located on the left half of the front control panel of base 56. The control pad has momentary contact membrane buttons that depress when pressed with at least 10 oz_{f} (or possibly another force). The buttons give a tactile “click” when they are pressed. The control pad is mounted at a 45° angle from vertical (or possibly a different angle). The following is one embodiment of the control buttons. Three buttons grouped together, labeled “Start/Pause Dialysis”, “Confirm Addition of Chemicals”, and “Silence or Reset an Alarm”. Nine additional buttons are grouped together, labeled: “Start Tank Rinse”, “Cancel Dialysis”, “Lock/Unlock Dialysis Program”, “Adjust Audio Volume”, “Set Clock”, “Set Desired Bed Time”, “Set Number of Nightly Cycles”, “Set Infusion Volume/Cycle”, and “Set Volume of Final Infusion”. Ten additional buttons are grouped together, labeled “0” through “9”.

A nephrologist or a renal nurse will initially set the number of dialysis cycles per night, the dialysate infusion volume for each dialysate exchange cycle (other than the final exchange cycle), and the dialysate volume in the final dialysate exchange cycle. To do this, the “Lock/Unlock Dialysis Program” button is pressed and a code number is entered. Then the “Set Number of Nightly Cycles” button is pressed, followed by entering the appropriate number. The “Set Infusion Volume/Cycle” button is then pressed, followed by entering the appropriate number. Finally, the “Set Volume of Final Infusion” button is pressed, followed by entering the appropriate number. The control software will not accept unreasonably high or low values for any of these parameters. Once all desired entries have been made, the “Lock/Unlock Dialysis Program” button is pressed.

The patient, a technician, a renal nurse, or a nephrologist will initially enter the patient's desired bed time. This is the time that the required volume of warm, sterile dialysate will be ready for use each evening. To do this, the “Set Desired Bed Time” button is pressed, the appropriate time is entered, and the “Set Desired Bed Time” button is pressed once again. If the machine's digital clock is displaying the incorrect time-of-day, it can be corrected by pressing the “Set Clock” button, entering the correct time, and then pressing the “Set Clock” button once again.

As seen in FIGS. 1, 5 and 6, base 56 contains electrical and other components, and fluid plumbing. The base also serves as a foundation for boiling vessel 1, fluid storage tank 12, cassette 30, boiling vessel demineralization system 44, tank rinsing system 46, LCD display 54, and control panel 55. Its outer dimensions are approximately 18″ wide×23″ deep×4.5″ high (or possibly different dimensions). The top panel of the base can be removed to conduct maintenance, repairs, or upgrades of the internal components. Some or all of the electrical components in the base may be coated or potted, to resist infiltration by fluids.

Cooling fan 57 is located in the left panel of base 56, near dialysate pump motor 58. The dialysate pump motor generates the most heat of all components in the base. The cooling fan is very quiet, generating about 14 dB (or possibly another sound volume).

As seen in FIG. 6, dialysate pump motor and control board 58 are mounted to the underside of the top of base 56, near the left front. In one embodiment, the motor drives dialysate pump rotor 39, which rotates clockwise or counterclockwise, depending on the operational stage. The control board keeps the motor rotation speed at 215 RPM (or possibly a different RPM), regardless of the load on the dialysate pump rotor. In an alternate embodiment, the pump motor drives a different type of dialysate pump mechanism, other than a rotor. The motor can deliver 150 inch-oz_{f} of torque (or possibly a different torque). The dialysate pump motor operates very quietly.

As seen in FIG. 6, cassette valve motor 59 is mounted to the underside of the top of base 56, next to dialysate pump motor and control board 58. The valve motor is attached to a set of reduction gears, which are in turn attached to cassette valve actuator 40. The valve motor reduction gear output shaft rotates 90 degrees in either direction, taking about two seconds to rotate a quarter-turn (about 7.5 RPM, or possibly a different rotational speed). The motor can deliver 42 inch-oz_{f} of torque (or possibly a different torque), measured at the reduction gear output shaft. The cassette valve motor operates very quietly. In an alternate embodiment, the cassette valve is not required, and therefore the cassette valve motor does not exist.

As seen in FIG. 6, audio message circuit board 60 generates pre-recorded, situation-specific verbal messages, when prompted by control and input/output circuit boards 83. The message files might be in the mp3 format. The two message types are instructional or alarm. Any message that is broadcast by speaker 61, is simultaneously displayed on LCD display screen 54. Audio alarm messages will be repeated until the patient takes the appropriate action(s), then presses the “Silence or Reset an Alarm” button on the control pad. An audio amplifier might be an integral part of the audio message circuit board, or it might be a separate circuit board. In an alternate embodiment, the audio message circuit board does not exist.

In one embodiment, when no messages are being generated, the following operational information is continuously displayed on the LCD display screen:
The time and date
The programmed patient bed time
The programmed number of nightly dialysis exchange cycles
The programmed dialysate infusion volume for each exchange cycle other than the final cycle
The programmed dialysate infusion volume for the final exchange cycle
The volume of dialysate that will be generated each day
The time that distillation will automatically begin each day
The audio volume setting
The volume of excess water removed from the patient by the previous night's dialysis
The operational step currently being conducted (paused, idling, distilling, cooling, adding chemicals, demineralizing the boiling vessel, sterilizing the dialysate, evacuating dialysate from the patient, infusing dialysate into the patient, dwell period, rinsing the fluid storage tank)
The temperature of the water or dialysate in the fluid storage tank (if any fluid is in the tank)
The electrical resistivity of distilled water in the fluid storage tank (if any water is in the tank)

When an instructional message is generated, the operational information that is normally displayed on the LCD display screen is temporarily replaced with the message. The message is also stated verbally. Some messages will be verbally repeated until the patient has completed the instruction. In one embodiment, instructional messages include the following:
Check that both water flow rates are at 250 IA/minute, and adjust them if needed
Check to see if the demineralization solution container needs filling, and fill it if needed
Perform maintenance activity “X” in “Y” days
Enter one or more missing dialysis parameters
Add chemicals to the distilled water, attach a new plastic port cap, close the steel cap, then press the “Confirm Addition of Chemicals” button
The dialysate is ready for use. Connect the dialysate tube to your Tenckhoff catheter, then press the “Start/Pause Dialysis” button.
Dialysis is complete. Disconnect the dialysate tube from your Tenckhoff catheter, connect the dialysate tube to the dialysate drain port, then press the “Start Tank Rinse” button
Replace the cassette and the tank rinse water container assembly with fresh ones

When an alarm message is generated, the operational information that is normally displayed on the LCD display screen is temporarily replaced with the message. The message is also stated verbally. The alarm messages will be verbally repeated until the patient takes an appropriate action. In one embodiment, alarm messages include the following:
Wake up please. Check for a kink in the dialysate tube. Straighten out the kink, then press the “Silence or Reset an Alarm” button.
A problem has been detected with the (fluid mixer motor, or demineralization solution pump motor, or tank rinse water pump motor, or dialysate pump motor, or demister UV lamp, or condenser UV lamp, or tank UV lamp, or boiling vessel heating element). Please press the “Silence or Reset an Alarm” button, call the Hotline phone number, and dialyze using CAPD until the problem is repaired or the machine has been replaced.
There is an insufficient amount of water in the boiling vessel. Press the “Silence or Reset an Alarm” button, then check to see why tap water is not flowing to the boiling vessel.
The distilled water is insufficiently purified. Please press the “Silence or Reset an Alarm” button, call the Hotline phone number, and dialyze using CAPD until the problem is repaired or the machine has been replaced.
The tank UV light is not outputting enough light. Please press the “Silence or Reset an Alarm” button, then replace the UV lamp.
A cassette has not been installed for too long. Please install a fresh cassette immediately.

As seen in FIG. 6, speaker 61 is mounted in base 56, near the right front side. A wire mesh in the base's right panel allows sound generated by the speaker to exit to the base. The speaker produces the verbal messages that are generated by audio message circuit board 60. In an alternate embodiment, the speaker does not exist.

As seen in FIG. 6, water electrical resistivity circuit board 62 is connected to water electrical resistivity sensor 24, which is mounted in the bottom wall of fluid storage tank 12. The sensor and circuit board continuously monitor the electrical resistivity of the distilled water in fluid storage tank 12, during the distillation and cool down phases. The circuit board energizes the sensor only after the distilled water level is high enough to cover the probe, and it de-energizes the sensor just before the dialysate chemicals are added to the distilled water. Distilled water of high purity has a high electrical resistivity, and vice versa. In addition, the electrical resistivity of a given sample of purified water is a function of its temperature. The electrical resistivity of the distilled water generated by the present invention is at least 1.0 Mohm-cm at 175° F., 3.0 Mohm-cm at 120° F., and 5.0 Mohm-cm at 98.6° F. In the present invention, the water resistivity alarm logic simultaneously considers both the measured resistivity and the temperature of the distilled water, on a continuous basis. An alarm signal is generated if the resistivity of the distilled water falls below the alarm resistivity limit that is appropriate for the temperature. The water electrical resistivity circuit board and the sensor are optional components. In an alternate embodiment, the electrical resistivity circuit board does not exist.

As seen in FIG. 6, power supply 63 is mounted in base 56. It is a 160 Watt (or possibly a different power) medical-grade power supply. The power supply provides 12 Volt DC power (or possibly a different voltage) to various electronic components in the machine. The power supply is electrically isolated for patient safety.

As seen in FIG. 6, DC-DC converter 64 is mounted in base 56. It converts 12 Volt DC current into 28 Volt DC current (or possibly a different voltage). The DC-DC Converter provides 28 Volt DC power (or possibly a different voltage) to various electronic components in the machine.

As seen in FIG. 6, boiling vessel demineralization solution pump motor 65 drives boiling vessel demineralization solution pump 43. The motor is located directly below the pump, at the top face of base 56. In one embodiment, the motor is attached to a set of reduction gears, which are in turn attached to the boiling vessel demineralization solution pump rotor. The speed of the motor can vary slightly with the load on the pump. In an alternate embodiment, the pump motor drives a different type of pump, other than a rotor. The motor delivers 22 inch-oz_{f} of torque (or possibly a different torque), measured at the reduction gear output shaft. The boiling vessel demineralization solution pump motor operates very quietly.

As seen in FIG. 6, tank rinse water pump motor 66 drives tank rinse water pump 45. The motor is located directly below the pump, at the top face of base 56. In one embodiment, the motor is attached to a set of reduction gears, which are in turn attached to the tank rinse water pump rotor. The speed of the motor can vary slightly with the load on the pump. In an alternate embodiment, the pump motor drives a different type of pump, other than a rotor. The motor delivers 22 inch-oz_{f} of torque (or possibly a different torque), measured at the reduction gear output shaft. The tank rinse water pump motor operates very quietly. In an alternate embodiment, the tank is never rinsed, so the water pump motor does not exist.

As seen in FIG. 6, ballasts 67, 68, and 69 power demister UV lamp 6, condenser UV lamp 9, and tank UV lamp 27. The ballasts are powered by 115V power (or possibly a different voltage). Because the first two UV lamps are optional components, their corresponding ballasts are optional components as well. In an alternate embodiment, the first two ballasts do not exist.

As seen in FIG. 6, current sensor for fluid mixer motor 70 continuously monitors the current drawn by fluid mixer motor 11 when it is energized. An alarm is generated if the current goes outside its alarm limits. This sensor is an optional component.

As seen in FIG. 6, current sensor for boiling vessel demineralization solution pump motor 71 continuously monitors the current drawn by boiling vessel demineralization solution pump motor 43 when it is energized. An alarm is generated if the current goes outside its alarm limits. This sensor is an optional component.

As seen in FIG. 6, current sensor for tank rinse water pump motor 72 continuously monitors the current drawn by tank rinse water pump motor 45 when it is energized. An alarm is generated if the current goes outside its alarm limits. This sensor is an optional component. In an alternate embodiment, the tank is never rinsed, so the current sensor for the tank rinse water pump motor does not exist.

As seen in FIG. 6, current sensor for dialysate pump motor 73 continuously monitors the current drawn by dialysate pump motor 58 when it is energized. An alarm is generated if the current goes outside its alarm limits. This sensor is an optional component.

As seen in FIG. 6, UV lamp current sensors 74, 75 and 76 continuously monitor the currents drawn by demister UV lamp 6, condenser UV lamp 9, and tank UV lamp 27, when the lamps are energized. An alarm is generated if any of these currents go outside their alarm limits. These sensors are optional components. In an alternate embodiment, the first two UV lamp current sensors do not exist.

As seen in FIG. 6, current sensor for boiling vessel heating element 77 continuously monitors the current drawn by boiling vessel heating element 3 when it is energized. An alarm is generated if the current goes outside its alarm limits. This sensor is an optional component.

As seen in FIG. 6, constant level tube solenoid valve 78 is a normally-open, intermittent duty solenoid valve that closes during the nightly demineralization of boiling vessel 1 and constant level tube 2, after distillation has been completed. Closing this valve allows the boiling vessel to be filled with demineralization solution as high as the boiling vessel's junction with the demister.

As seen in FIG. 6, boiling vessel drain solenoid valve 79 is located in base 56, near boiling vessel drain port 50. The valve is a normally-closed, intermittent duty solenoid valve that is opened when boiling vessel 1 needs to be drained.

As seen in FIG. 6, condenser water solenoid valve 80 is located in base 56, near condenser water flow controller 48. The valve is a normally-closed, continuous duty solenoid valve that opens to allow tap water to flow through the condenser during distillation. This valve is opened each afternoon at the start of distillation, and it is closed when the required volume of tap water has been distilled.

As seen in FIG. 6, tank cooling water solenoid valve 81 is located in base 56, near tank cooling water flow controller 47. The valve is a normally-closed, continuous duty solenoid valve that opens to allow tap water to flow through tank cooling water pad 25. This valve is opened each afternoon at the start of distillation, and it is closed when the required volume of tap water has been distilled and cooled to 98.6° F.

As seen in FIG. 6, power entry module 82 is located in the rear panel of base 56. It includes a ground circuit and two 120 Volt, 15 Amp (or possibly a different current) in-line fuses, that are easily reachable and replaceable. The module's connector type is IEC 60320-1-C14 (or possibly a different type).

As seen in FIG. 6, control and input/output circuit boards 83 are located in base 56. The control circuit board might incorporate a software operating system (OS). If a software operating system is used, it can be a real-time embedded OS (or possibly a different type). The high-level software language can be “C” or “C++” (or possibly a different language).

The following components give electronic input to control and input/output circuit boards 83. Some of these components are for safety, system checks or dialysate quality checks only, and may therefore be considered to be optional components. Although only two point level sensors are listed below, four or five point level sensors may be required.

| | |
|---|---|
| Boiling vessel temperature sensor | 4 |
| Tank UV light sensor | 16 |
| Tank upper point level sensor | 18 |
| Tank lower point level sensor | 19 |
| Heating pad temperature sensor | 23 |
| Tank fluid temperature probe | 26 |
| Cassette dialysate pressure sensor | 36 |
| Buttons on the control pad | 55 |
| Water electrical resistivity circuit board | 62 |
| Current sensor for the fluid mixer motor | 70 |
| Current sensor for boiler demineralization sol. pump motor | 71 |
| Current sensor for the tank rinse water pump motor | 72 |
| Current sensor for the dialysate pump motor | 73 |
| Current sensor for the demister UV lamp | 74 |
| Current sensor for the condenser UV lamp | 75 |
| Current sensor for the tank UV lamp | 76 |
| Current sensor for the boiling vessel heating element | 77 |

The following electronic components receive control output from control and input/output circuit boards 83. Some of these components are for safety, system checks or dialysate quality checks only, and may therefore be considered to be optional components.

| | |
|---|---|
| Boiling vessel heating element | 3 |
| Fluid mixer motor | 11 |
| Tank chemical fill port locking mechanism | 13 |
| Tank heating pad | 22 |
| LCD display screen | 54 |
| Dialysate pump motor and control board | 58 |
| Cassette valve motor | 59 |
| Audio message circuit board | 60 |
| Water electrical resistivity circuit board | 62 |
| Boiling vessel demineralization solution pump motor | 65 |
| Tank rinse water pump motor | 66 |
| Demister UV lamp ballast | 67 |
| Condenser UV lamp ballast | 68 |
| Tank UV lamp ballast | 69 |
| Constant level tube solenoid valve | 78 |
| Boiling vessel drain solenoid valve | 79 |
| Condenser water solenoid valve | 80 |
| Tank cooling water control solenoid valve | 81 |

The control software in the control board has the following capabilities:
Generate the information listed in paragraph [0085] above, and display it on the LCD display screen
Generate the situation-specific instructional messages listed in paragraph [0086] above as needed, on the LCD display screen and verbally
Generate the situation-specific alarm messages listed in paragraph [0087] above as needed, on the LCD display screen and verbally
Maintain a perpetual countdown to the next due dates for all periodic maintenance tasks
Compare inputs from the following sensors against appropriate alarm limits, and stop the machine and generate an alarm if appropriate: the boiling vessel temperature sensor, the tank UV light intensity sensor, the electrical current sensors for eight components, the distilled water electrical resistivity sensor, and the length of absence of an installed cassette
Calculate the required distillation start time based on the patient's programmed bed time and the total time required to complete the following tasks: distill the required volume of water, cool the distilled water to 98.6° F., add and mix chemicals into the distilled water, and sterilize the dialysate using UV light
De-energize the boiling vessel heating element when the required volume of distilled water has been distilled
Shut off the tank cooling water when the distilled water has been cooled to 98.6° F.
Control the tank heating pad such that the dialysate temperature does not drop below 98.6° F.
Rinse the boiling vessel with the correct volumes of demineralizing fluid and tap water
When the distilled water reaches 98.6° F., energize the mixer and prompt the patient to introduce chemicals to the tank
When the “Confirm Addition of Chemicals” button has been pressed, lock the chemical fill port cap and de-energize the mixer after one minute (or possibly a different elapsed time)
When the “Confirm Addition of Chemicals” button has been pressed, de-energize the tank UV lamp after 40 minutes (or possibly a different elapsed time)
Calculate the required pumping times and the required dwell times between each exchange cycle, based on the total nightly volume of dialysate prescribed by the nephrologist
Calculate the required pumping times and the required dwell times between each exchange cycle, based on the programmed number of dialysate exchange cycles and dialysate infusion volumes
Alert the patient when dialysis is ready to begin, and begin the dialysate exchange cycle program after the “Start/Pause Dialysis” button has been pressed
Control the dialysate infusion pumping and the spent dialysate evacuation pumping
Monitor the pressure in the dialysate tube, and react if a substantial pressure drop is sensed
Use the elapsed pumping times to calculate the excess fluid removed from the patient for each exchange cycle, and for the nightly cumulative total
When dialysis has been completed, alert the patient to disconnect from the dialysate tube
When the “Start Tank Rinse” button has been pressed, rinse the tank with distilled water
After the tank rinse has been completed, alert the patient to replace the cassette and the tank rinse water container assembly

Dextrose is currently the world-standard chemical in peritoneal dialysate, for inducing the shedding of excess water in dialysis patients. The chemical name for dextrose is Glucose Monohydrate. To be used in a dialysate, dextrose is processed to have low endotoxin and bioburden contents. The dextrose is in powder form, which aids in dissolving it quickly in water. In the present invention, a weighed quantity of dextrose is packaged in plastic daily-dose bottles. The bottle opening is designed to mate with the tank chemical fill port. The amount of dextrose is appropriate for the patient's body size, kidney health, daily fluid intake, and peritoneum transport speed. The amount will vary from about 91.4 grams (for 6 liters of 1.5% dialysate per day) to about 798.9 grams (for 18 liters of 4.25% dialysate per day).

Peritoneal dialysate contains specific concentrations of sodium chloride, magnesium chloride, and calcium chloride. To be used in a dialysate, these salts are processed to have low endotoxin and bioburden contents. In the present invention, the salts are in powder form, which aids in dissolving them quickly in water. Weighed quantities of these salts are packaged together in small plastic daily-dose bottles. The bottle opening is designed to mate with the tank chemical fill port. The amount of each salt in a bottle is appropriate for the patient's required daily volume of dialysate, as well as his particular blood chemistry at the time. For patients with healthy blood chemistry, the total daily amount of salts vary from about 35.1 grams (for 6 liters of dialysate per day) to about 102.3 grams (for 18 liters of dialysate per day). A slightly greater or lesser amount of sodium chloride or calcium chloride might be included in order to accommodate a patient who has hypo or hypercalcemia, or hypo or hypernatremia.

Sodium lactate is often used in peritoneal dialysate as a buffer. To be used in a dialysate, sodium lactate is processed to have low endotoxin and bioburden contents. In the present invention, a measured quantity Sodium lactate is packaged in small plastic daily-dose bottles. The bottle opening is designed to mate with the tank chemical fill port. The daily amount is in proportion to the patient's required daily volume of dialysate. This varies from about 26.9 grams of pure Sodium Lactate (for 6 liters of dialysate per day) to about 80.6 grams (for 18 liters of dialysate per day). The sodium lactate can be in solution form or powder form.

The numerical values and ranges in this document that specify mass, voltage, pH, pressure, volume, flow rate, etc., have been given as precisely as presently possible. However, unless otherwise indicated, all numbers and ranges specified in this document are to be understood as being modified by the term “about”. Ranges of values herein are intended to serve as a shorthand method of referring individually to each separate value falling within the range. Unless otherwise indicated herein, each individual value is incorporated into the specification as if it were individually recited herein.

The terms “a” and “an” and “the”, and similar referents used in this document are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified.

Preferred embodiments of this invention are described herein, including the best mode known to the inventor for carrying out the invention. Of course, upon reading the foregoing description, variations on those preferred embodiments will become apparent to those of ordinary skill in the art. This invention includes all modifications and equivalents of the subject matter recited in the claims as permitted by applicable law. Moreover, any combination of the above-described elements in all possible variations thereof is encompassed by the invention unless otherwise indicated herein or otherwise clearly contradicted by context.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A peritoneal dialysis system comprising: a boiling vessel that generates steam; a condenser that condenses the steam; a fluid storage vessel that collects the condensate; an ultraviolet lamp that sterilizes the fluid in the fluid storage vessel; a fluid mixer; a dialysate cassette; and a dialysate pump.

2. The peritoneal dialysis system of claim 1, further comprising a boiling vessel constant fluid level mechanism, which includes an automatically-controlled valve.

3. The peritoneal dialysis system of claim 1, further comprising an automatically-controlled boiling vessel drain valve.

4. The peritoneal dialysis system of claim 1, further comprising a boiling vessel demineralization system.

5. The peritoneal dialysis system of claim 1, further comprising a demister that is configured such that water vapor flows from the boiling vessel, through the demister, and into the condenser.

6. The peritoneal dialysis system of claim 1, further comprising an ultraviolet lamp that is in close proximity to the condenser.

7. The peritoneal dialysis system of claim 1, further comprising a water flow controller and an automatically-controlled valve for the water flowing to the condenser.

8. The peritoneal dialysis system of claim 1, further comprising a quartz tube mounted in the fluid storage vessel, which prevents fluids from contacting the ultraviolet lamp in the fluid storage vessel.

9. The peritoneal dialysis system of claim 1, further comprising the following sensors located in or on the fluid storage vessel: one or more fluid level sensors; a fluid temperature sensor.

10. The peritoneal dialysis system of claim 1, further comprising the following sensors located in or on the fluid storage vessel: a fluid electrical resistivity sensor; an ultraviolet light sensor.

11. The peritoneal dialysis system of claim 1, further comprising heating apparatus for the fluids in the fluid storage vessel.

12. The peritoneal dialysis system of claim 1, further comprising a cooling apparatus for the fluids in the fluid storage vessel, which includes a water flow controller and an automatically-controlled valve.

13. The peritoneal dialysis system of claim 1, further comprising a fluid storage vessel rinsing system.

14. The peritoneal dialysis system of claim 1, further comprising a dialysate filter that filters dialysate flowing from the fluid storage vessel.

15. The peritoneal dialysis system of claim 1, further comprising a flexible tube that routes pressurized tap water to the peritoneal dialysis system, and a second flexible tube that routes spent fluids from the peritoneal dialysis system to a toilet or a sewer drain.

16. The peritoneal dialysis system of claim 1, further comprising a display screen that displays operational information and/or instructions and/or warnings.

17. The peritoneal dialysis system of claim 1, further comprising an audio message system that produces verbal messages that give operational information and/or instructions and/or warnings.

18. The peritoneal dialysis system of claim 1, further comprising current sensors that measure the electrical current drawn by the boiling vessel heating element, and/or the fluid mixer motor, and/or the fluid storage vessel ultraviolet lamp, and/or the dialysate pump motor.

19. The peritoneal dialysis system of claim 1, further comprising a base that houses fluid and electronic components, and that also supports assemblies mounted to its top surface, including a cassette holding fixture.

20. A method of generating peritoneal dialysate and conducting peritoneal dialysis, comprising the following steps:
a. boiling water to produce steam;
b. condensing the steam to produce distilled water;
c. cooling the distilled water to approximately body core temperature;
d. mixing electrolyte salts, low-endotoxin dextrose, and low-endotoxin sodium lactate into the distilled water, to produce dialysate;
e. sterilizing the dialysate by exposing it to ultraviolet radiation;
f. pumping substantially all spent peritoneal dialysate from the patient's peritoneal cavity to a drain, via a flexible tube;
g. pumping a predetermined volume of dialysate into the patient's peritoneal cavity via the same flexible tube as in step “f” above;
h. confining the dialysate in the patient's peritoneal cavity for a predetermined time period;
i. pumping substantially all spent peritoneal dialysate from the patient's peritoneal cavity to a drain, via the same flexible tube as in step “f” above;
j. repeating the above three steps “g”, “h”, and “i” a predetermined number of times;
k. pumping a predetermined volume of dialysate into the patient's peritoneal cavity via the same flexible tube as in step “f” above;
l. confining the dialysate in the patient's peritoneal cavity throughout part or all of the following day.

21. The method of claim 20, wherein the dialysate is filtered as it is pumped into the patient's peritoneal cavity.
